(19) United States
(12) Patent Application Publication
(10) Pub. No.: US 2024/0110853 A1
(43) Pub. Date: Apr. 4, 2024

(54) METHOD FOR OPTICALLY CLEARING A TISSUE SAMPLE USING AN EMBEDDING MEDIUM

(71) Applicant: MobiCron GmbH, Göttingen (DE)

(72) Inventors: Stephan Diekmann, Diessen (DE); Fred S. Wouters-Bunt, Göttingen (DE)

(21) Appl. No.: 18/257,833

(22) PCT Filed: Dec. 15, 2021
(86) PCT No.: PCT/EP2021/085982

§ 371 Date: Jun. 15, 2023

(30) Foreign Application Priority Data
Dec. 17, 2020 (EP) 20215095.9

(51) Int. Cl. G01N 1/30; G01N 1/34; G01N 1/36

(52) U.S. Cl. CPC G01N 1/30; G01N 1/34; G01N 1/36

(57) ABSTRACT

The invention relates to a method for producing transparent tissue samples of a biological or human tissue for a light microscopy examination, having the steps a) dewatering the tissue sample using a dewatering solvent and b) clearing the dewatered tissue sample by transferring same into an embedding medium which contains an benzaldehyde-anisole ether. In comparison to the benzyl benzoate/benzyl alcohol mixtures and methyl salicylate-benzyl benzoate mixtures conventionally used until now as embedding media, the benzaldehyde-anisole ether has the advantage of being usable as a pure substance, in exactly the same way as a dibenzyl ether: Because the pure substance already has the desired refractive index, the refractive index does not need to be set by mixing the embedding medium. Furthermore, the benzaldehyde-anisole ether according to the invention penetrates the dewatered tissue faster than previously known embedding media and also makes the tissue transparent faster.

### RELATED APPLICATION INFORMATION

This patent claims priority from International PCT Patent Application No. PCT/EP2021/085982, filed Dec. 15, 2021 entitled, “METHOD FOR OPTICALLY CLEARING A TISSUE SAMPLE USING AN EMBEDDING MEDIUM”, which claims priority to European Patent Application No. 20215095.9, filed Dec. 17, 2020, all of which are incorporated herein by reference in their entirety.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

The invention relates to a method according to the preamble of claim 1, to a kit for preparing biological tissue samples as claimed in claim 10, and to a use as claimed in claim 13.

Transparent biological tissue samples are needed in order for tissue samples to be able to undergo three-dimensional imaging, for example by light-sheet microscopy. The achievement of transparency in biological preparations necessitates the removal from the biological preparations of heme groups of the blood pigment hemoglobin and lipids in particular. In the “dehydrating” processes, the tissue is treated with various mixtures of a water-miscible organic solvent and water. The treatment is performed with an increasing proportion of the organic solvent in order to completely remove the water from the tissue. Here there are a number of options, for example tetrahydrofuran, methanol, isopropanol, tert-butanol, and ethanol. Ethanol is currently the most commonly used dehydration medium for tissue clearing in pathology. The end result of all “dehydrating” processes is an anhydrous sample.

The final step in the various clearing methods is the adjustment of the refractive index to the refractive index of the tissue undergoing microscopy. The refractive index of dehydrated tissue is according to Spalteholz (“Über das Durchsichtigmachen von menschlichen and tierischen Präparaten” [On the transparency of human and animal preparations], published by S. Hirzel, 1911, German Reich Patent No. 229044) n=1547 for bone. The refractive index of other tissues can be estimated at approx. n=1.551 based on the optimal mixtures that he determined empirically. Such a high refractive index requires the use of aromatic compounds, which are generally immiscible with water. For his investigations, Spalteholz used mixtures of wintergreen oil (methyl salicylate) and benzyl benzoate or isosafrole in a mixing ratio tailored to the various tissues.

A variant of the Spalteholz method is known from Dodt, Leischner, Schierloh, Jährling, Mauch, Deininger, Deussing, Eder, Zieglgänsberger, and Becker “Ultramicroscopy: three-dimensional visualization of neuronal networks in the whole mouse brain”. Nat Methods. 2007; 4(4): 331-6.), in which “Murray's clear” (a 2:1 mixture of benzyl benzoate and benzyl alcohol with a refractive index of n=1.559) is used as the embedding medium. Another variant is known from Becker, Jährling, Saghafi, Weiler, and Dodt “Chemical Clearing and Dehydration of GFP Expressing Mouse Brains” (2012) PLoS ONE 7(3): e33916. https://doi.org/10.1371/journal.pone.0033916. Spalteholz embedding media and a number of other aromatic compounds are tested. Dibenzyl ether (DBE, refractive index n=1.562) emerged as the most suitable compound and has become the de facto gold standard in tissue clearing. The advantage of DBE is also that it is easy to use because the solution does not need to be mixed and its refractive index then checked; it can be used directly in the form of the pure substance.

From WO 2017/093323 A1 the use of ethyl cinnamate (ECi) and related cinnamyl esters is known as non-toxic alternatives to the embedding media in common use up till then. It exhibits lower acute and chronic toxicity than the other aromatic compounds used for tissue treatment, such as dibenzyl ether, benzyl alcohol or benzyl benzoate. Since as a pure substance it achieves the desired refractive index, a mixing step is eliminated and thus a work step and a possible source of error.

The selection of pure liquid substances that are suitable as embedding media for dehydrated samples is thus currently limited to the two compounds dibenzyl ether and ethyl cinnamate. However, both substances have drawbacks: dibenzyl ether is toxic. Although ethyl cinnamate has low toxicity, its melting point is 6-9° C., so samples cleared with ethyl cinnamate cannot be stored in a refrigerator because the embedding medium will crystallize there. Ethyl cinnamate also has a comparatively high vapor pressure.

The object of the invention is to provide a method for preparing a transparent tissue sample of a biological tissue for examination by light microscopy that overcomes the above disadvantages and that in particular involves less effort than having to produce mixtures as the embedding medium, avoids embedding media with high toxicity, and permits the refrigerated storage of the dehydrated tissue samples.

The object is achieved according to the invention by a method as claimed in claim 1. The object is additionally achieved by a kit as claimed in claim 9 and by the use as claimed in claim 11.

Further embodiments are the subject matter of the dependent claims or described below.

The method according to the invention for preparing transparent tissue samples of a biological tissue for examination by light microscopy comprises the steps of:
a) dehydrating the tissue sample with a dehydrating solvent and
b) clearing the dehydrated tissue sample by placing it in a liquid embedding medium having a refractive index that matches the tissue.

The embedding medium comprises a benzaldehyde anisole ether, preferably selected from 3-methoxybenzaldehyde, 4-methoxybenzaldehyde, 2-hydroxy-5-methoxybenzaldehyde, and 4-ethoxybenzaldehyde.

The purpose of the method according to the invention is to achieve optical transparency in a biological or human tissue sample for light microscopy. The biological tissue is for example human tissue or animal tissue.

The clearing step of the clearing method is the adjustment of the refractive index to the refractive index of the tissue undergoing microscopy. For this, the tissue sample is transferred to a solution for adjustment of the refractive index: the embedding medium. The embedding medium must be miscible with the solvent used in the dehydration step.

In one embodiment, the embedding medium contains 10% to 100% by volume of the benzaldehyde ether and 0% to 90% by volume of an optically suitable, inert organic solvent having a refractive index of about 1.3, preferably about 1.5. For example, the embedding medium contains 90% to 100% by volume of the benzaldehyde ether.

In another embodiment, the embedding medium contains 10% to 100% by volume of the benzaldehyde ether and 0% to 90% by volume of an optically suitable, inert organic solvent having a refractive index of about 2.0, preferably about 1.65. For example, the embedding medium contains 90% to 100% by volume of the benzaldehyde ether.

All percentages according to the invention are percentages by volume (vol %).

In the preferred embodiment, the embedding medium consists of a benzaldehyde anisole ether of the invention, i.e. the benzaldehyde anisole ether is used as the pure substance. The pure substance is here understood as meaning the benzaldehyde anisole ether in the technically available purity.

The benzaldehyde ether is a benzaldehyde anisole ether. The benzaldehyde anisole ether is preferably selected from 3-methoxybenzaldehyde (meta-anisaldehyde; CAS No. 591-31-1), 4-methoxybenzaldehyde (para-anisaldehyde; CAS No. 123-11-5), 2-hydroxy-5-methoxybenzaldehyde (6-hydroxy-m-anisaldehyde; CAS No. 672-13-9), and 4-ethoxybenzaldehyde (homoanisaldehyde; CAS No. 10031-82-0). 3-Methoxybenzaldehyde as the technical product has a purity of approx. 97.0%. 4-Methoxybenzaldehyde as the technical product has a purity of approx. 97.0%. 2-Hydroxy-5-methoxybenzaldehyde as the technical product has a purity of approx. 98.0%. 4-Ethoxybenzaldehyde as the technical product has a purity of approx. 99%.

The purpose of the dehydration step a) is to obtain a water-free tissue sample. The tissue sample is in one embodiment preferably treated in a number of passes with various dehydrating compositions in a decreasing water series, i.e. mixtures containing an increasing proportion of a water-miscible organic solvent, to remove the water from the tissue. As dehydrating medium alcohols, ketones or ethers are used. Suitable dehydrating solvents are for example ethanol, methanol, isopropanol, tert-butanol (IUPAC: 2-methylpropan-2-ol), tetrahydrofuran or acetone.

The dehydration medium here has the following properties: 1. it is completely miscible with water in order that the water and the fixative can be gradually removed from the tissue by means of an increasing concentration series, and 2. it is completely miscible with the embedding medium with which the refractive index is adjusted to that of the dehydrated tissue.

In an alternative embodiment, the dehydration step a) is carried out with 2,2-dimethoxypropane (DMP), which removes said water from the tissue by a chemical reaction with the water present in the tissue.

In the method according to the invention, the dehydration step a) to obtain a water-free tissue sample involves in one embodiment the use of dehydrating compositions
composed of aqueous ethanol having increasing concentrations of ethanol, wherein the ethanol concentrations of the dehydrating compositions range from 30% to 100% by volume, or
composed of aqueous tetrahydrofuran having increasing concentrations of tetrahydrofuran, wherein the tetrahydrofuran concentrations of the dehydrating compositions range from 30% to 100% by volume, or
composed of aqueous methanol having increasing concentrations of methanol, wherein the methanol concentrations of the dehydrating compositions range from 30% to 100% by volume, or
composed of an aqueous mixture of another alcohol, ketone or ether, wherein the solvent concentration of the dehydrating compositions ranges from 30% to 100% by volume.

In an alternative embodiment of the method according to the invention, the dehydration step a) to obtain a tissue sample having a residual water content of >2% is carried out using dehydrating compositions
composed of aqueous ethanol having increasing concentrations of ethanol, wherein the ethanol concentrations of the dehydrating compositions range from 50% to 98% by volume, preferably 70% to 98%, more preferably 75% to 98% by volume, or
composed of an aqueous mixture of another alcohol, ketone or ether, wherein the solvent concentration of the dehydrating compositions ranges from 50% to 98% by volume, preferably 70% to 98%, more preferably 75% to 98% by volume.

The dehydrating compositions used preferably, even in the final stage of the dehydration step still contain 2-30% by volume of water. In this embodiment it is also possible to use for example denatured ethanol having a residual water content of e.g. 4-6% by volume.

In a further alternative embodiment of the method according to the invention, the dehydration step a) is performed in a gradient mixer. For this, a tissue sample is placed in a mixing vessel (gradient mixer). A dehydrating solvent is introduced via an inlet until a residual water content of 5% to 20% by volume is reached. The dehydrating solvent may have a solvent concentration of 100% by volume or a residual water content of between 2% and 50% by volume. In one variant, the tissue sample in the gradient mixer is at the start introduced directly into a dehydrating solvent having a solvent concentration of 50% by volume and the gradient then increased in small increments.

The tissue sample is preferably transferred directly from the gradient mixer to the embedding medium without first being incubated in a high-purity solvent. Preferably no incubation step in a high-purity solvent takes place in step a) or step b).

It is surprising that clearing of the tissue samples in this way is possible. According to the prior art, the method in a gradient mixer proceeds as follows: A tissue sample is placed in a mixing vessel (gradient mixer). The vessel has for example an inlet at the top and a side outlet. If the sample in 50% ethanol is placed in this mixing vessel and at a high concentration (>95% by volume of solvent) is introduced with a dosing pump via the inlet, then a gradient builds up that attempts asymptotically to attain a value of 100% by volume. This results in the sample being supplied with an advantageous steadily increasing ethanol content. Since it is impossible to reach 100% with the mixture, the sample is normally subsequently incubated in high-purity ethanol. According to the prior art, this incubation step is repeated multiple times. Therefore, the dosing pump can for cost reasons be routinely filled with technical grade ethanol.

In the method of the invention, 1. the final (optionally repeated) incubation step with absolute ethanol has become superfluous and 2. the end point reached with the gradient mixer is sufficient for further clearing of the tissue sample in step b), provided the ethanol content is above about 80%.

In a further alternative embodiment, the dehydration step a) is performed in a single pass. A dehydration series comprising two or more passes is not used; instead, the dehydration according to step a) is carried out in a single pass by placing the tissue sample in dehydrating solvent having a solvent concentration of at least 70% by volume. This embodiment is particularly suitable for tissue samples with compact tissue that absorb little water. In the subsequent step b), the tissue sample is immediately placed in the embedding medium, again without incubation in a high-purity solvent for complete dehydration. This results in a significant time saving and at the same time also in significant savings on the reagents used. The savings on reagents result firstly from the smaller amount of solvent that is needed. Secondly, it is no longer necessary to use a high-purity anhydrous solvent; a technical grade solvent can instead be used. In the case of ethanol it is not necessary for example to use costly 100% high-purity ethanol; much less costly denatured ethanol can instead be used. According to the prior art, the incubation with high-purity anhydrous solvent is repeated in order to completely remove the last traces of water, with many protocols also repeating the incubation in the embedding medium for the same reason. Since the final incubations for reliable removal of residual water from the tissue are omitted in both embodiments of the invention, the method of the invention results in savings on reagents.

In one embodiment of the method according to the invention, the tissue sample, before it is dehydrated in step a) and optically cleared in step b), is
fixed and/or
fixed with formaldehyde and/or
washed and/or
washed with water and/or
incubated in an aqueous alkaline solution containing nonionic detergent and/or
delipidated with a detergent solution (delipidation) and/or
delipidated with an organic solvent (delipidation) and/or
bleached with oxidizing reagents and/or
decolorized with amino alcohols.

In one embodiment of the method according to the invention, the tissue sample, before it is dehydrated in step a) and optically cleared in step b), is fixed and the fixing agent is selected from
crosslinking fixatives such as formaldehyde, glutaraldehyde, acrolein, carbodiimides, diethyl pyrocarbonate, bisimidoesters or glyoxal or mixtures thereof, and/or
coagulant fixatives such as alcohols and other organic solvents, acids, potassium dichromate, lead nitrate, copper sulfate, and mercuric chloride and mixtures thereof.

The method according to the invention is preferably performed after tissue preparation steps and after electrophoresis steps. The tissue sample treated in the method according to the invention will thus already have been pretreated. The pretreatment by electrophoresis is preferably carried out according to the electrophoretic clearing method described in DE 10 2016 123 458 B3. For the performance of the electrophoretic clearing, reference is made to patent specification DE 10 2016 123 458 B3, the content of which is hereby incorporated into this application.

In the method according to the invention, the optically cleared tissue sample is preferably in a further step examined under a microscope in order to obtain an image of the internal structure of the sample, wherein the microscope is a light microscope, preferably a light-sheet microscope, a confocal microscope, a two-photon microscope or an optical projection tomography (OPT) microscope.

The embedding medium dibenzyl ether (DBE) used up to now has a refractive index of n=1.562. The second pure substance ethyl cinnamate has a refractive index of n=1.559. Of the benzaldehyde anisole ethers of the invention, 3-methoxybenzaldehyde has for example a refractive index of n=1.552 and 2-hydroxy-5-methoxybenzaldehyde a refractive index of n=1.580. Use of the benzaldehyde anisole ethers of the invention as an embedding medium for tissue clearing of dehydrated tissue samples achieved a transparency at least identical to, in some cases a transparency better than, that of tissue samples treated with methyl salicylate/benzyl benzoate and with ethyl cinnamate.

The studies by Spalteholz had previously suggested that the refractive indices of different tissues differ slightly from one other. His original 1911 publication lists a series of human tissues in order of increasing refractive index determined empirically based on the optimal mixing ratio of methyl salicylate and BB: young embryos (5:1-3:1 depending on weight)<adult decalcified bones (5:3) n=1.547<adult muscle approximately the same as older embryos (2:1)<brain and spinal cord (1:1). This variability has direct consequences for the transparency of different tissues and for the examination of said tissues by microscopy, and for quantitative spectroscopic determinations that can be performed with a microscope in particular. The method according to the invention offers the advantage that various pure substances are used as the embedding medium, which can be selected according to tissue type so as to approximate to the refractive index of the tissue.

Compared to the benzyl benzoate/benzyl alcohol mixtures (BABB) and methyl salicylate/benzyl benzoate mixtures typically used as embedding media up to now, the benzaldehyde anisole ethers have the advantage that, just like dibenzyl ether, they can be used in the form of the pure substance: Since the desired refractive index is already achieved by the pure substance, there is no need for the refractive index to be set through the mixing of the embedding medium. This eliminates a mixing step and thus a superfluous work step and a possible source of error.

Surprisingly, the benzaldehyde anisole ethers of the invention penetrate the dehydrated tissue much more rapidly than the embedding media known up to now and rapidly make the tissue transparent. This saves time when preparing the sample, allowing the sample to be provided for examination under the light microscope more swiftly. The results of the light microscopy examinations accordingly also become available more swiftly. The density of the dehydrated tissue is determined by the density of the dehydrating solvent, for example ethanol, still present in the tissue. Since the embedding medium typically has a higher density, the complete penetration of the embedding medium into the tissue sample can be easily monitored by the sinking of the tissue sample in the embedding medium. For example, with 2-hydroxy-5-methoxybenzaldehyde the embedding medium achieves complete penetration into the tissue sample after just 30-45 min, i.e. the tissue samples are lying on the bottom of the vessel. With conventional embedding media such as dibenzyl ether or methyl salicylate/benzyl benzoate mixtures, on the other hand, this method step typically takes several hours to overnight. The method according to the invention thus offers the advantage that the embedding medium diffuses more rapidly into the tissue of the tissue sample, achieving a significant time saving in the preparation of the sample. The tissue samples can thus be examined by light microscopy more swiftly.

If the tissue sample contains residual water and is transferred to an organic solvent as embedding medium in which this water can no longer dissolve, the water becomes trapped in the tissue and, because of its incomplete miscibility, results in turbidity that adversely affects the transparency. It has accordingly up to now been customary, for example, for the tissue samples to be incubated multiple times with high-purity ethanol and often subsequently repeatedly with embedding medium so as to avoid adversely affecting the transparency of the sample. According to the prior art, as has already been explained, the incubation with high-purity anhydrous solvent is repeated in order to completely remove the last traces of water, with many protocols also repeating the incubation in the embedding medium for the same reason.

The clearing of the tissue is according to the invention carried out with a benzaldehyde anisole ether. Surprisingly, it was found that good transparency can be achieved also with tissue samples that contain residual water. At small residual water contents of 2-5% by volume, no adverse effect on transparency is observed, and even at high water contents of up to 20% or up to 30% by volume, depending on the type of tissue treated, only very low turbidity that still allows examination of the sample by light microscopy is observed.

The method according to the invention therefore offers a particularly rapid and simple method for dehydrating a tissue sample, since, unlike all embedding media known up to now, a low residual water content of up to 10% by volume has no adverse effect on the results of light microscopy and acceptable transparency in the tissue sample is achieved even with larger amounts of residual water.

The method according to the invention also allows a simplified procedure compared to clearing methods using conventional embedding media. According to the prior art, the tissue sample is during dehydration transferred from vessel to vessel having increasing concentrations of the dehydrating solvent, for example having an increasing concentrations of ethanol. This necessitates quite a few individual steps, these being discrete steps. Moreover, the use of a high-purity solvent, for example high-purity ethanol, is always necessary in the final step(s).

In the method according to the invention it is also possible to use a dehydrating solvent that has a residual content of water. For example, denatured 95-97% technical grade ethanol can be used instead of high-purity ethanol. The method according to the invention therefore also results in savings on the costs of the solvents used.

The method according to the invention also permits a simplified procedure when using a gradient mixer, since there is likewise no need here for subsequent complete dehydration with a high-purity solvent.

The benzaldehyde anisole ethers moreover have low toxicity. This is advantageous when used as an embedding medium, since the tissue sample in the microscope is positioned immediately below the user's airways and the user can inhale the vapor rising from the sample. According to the European Chemicals Agency ECHA, the toxicity of para-anisaldehyde is not a toxicological concern, unlike that of dibenzyl ether or benzyl benzoate/benzyl alcohol mixtures.

The kit according to the invention for preparing biological tissue samples for light microscopy comprises:
a dehydrating solvent for dehydrating the tissue sample and
an embedding medium for clearing the dehydrated tissue sample by placing the sample in the embedding medium,
where
the embedding medium is a benzaldehyde anisole ether as described above, selected from 3-methoxybenzaldehyde, 4-methoxybenzaldehyde, 2-hydroxy-5-methoxybenzaldehyde, and 4-ethoxybenzaldehyde. The benzaldehyde anisole ether is preferably employed in the concentrations and purities described above.

The dehydrating solvent in the kit is an ether, ketone or alcohol, preferably the solvent is selected from ethanol, methanol, isopropanol, tert-butanol, 2,2′-thiodiethanol, trichloroethanol, tetrahydrofuran, and acetone.

According to the invention, a benzaldehyde anisole ether selected from 3-methoxybenzaldehyde, 4-methoxybenzaldehyde, 2-hydroxy-5-methoxybenzaldehyde, and 4-ethoxybenzaldehyde is used as an embedding medium for preparing a biological, in particular human, tissue sample for examination by light microscopy.

The benzaldehyde anisole ether used according to the invention is preferably used in the concentrations and purities described above.

### EXAMPLES

### Example 1

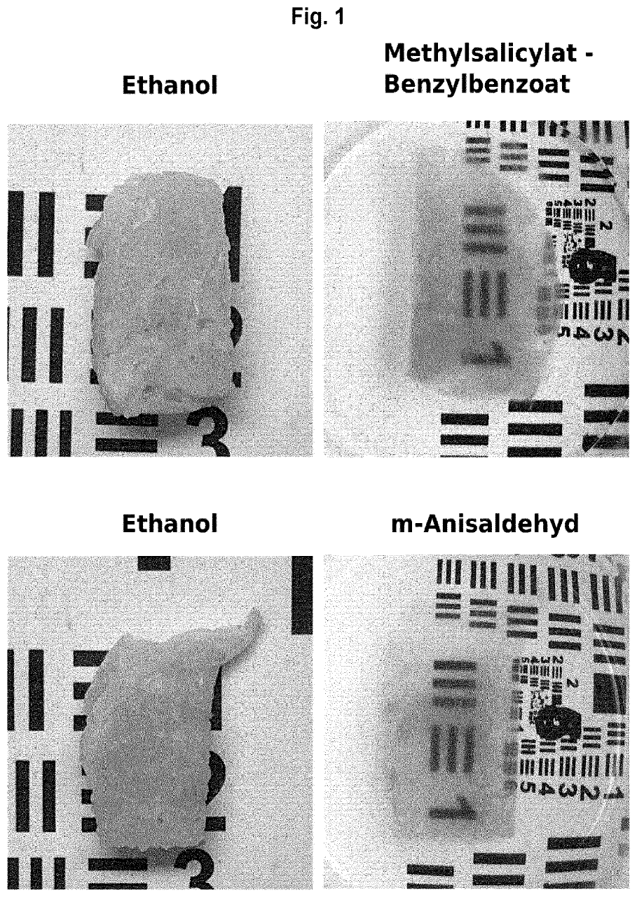

Several tissue samples (pig lung) having a volume of about 0.25 ml (0.5×0.5×1 cm 3) were dehydrated in absolute ethanol and photographed. The tissue samples dehydrated with ethanol are shown in FIG. 1, left-hand side.

The dehydrated tissue samples were then placed in 5 ml of the respective embedding medium for 3 hours. The embedding media used were 2-hydroxy-5-methoxybenzaldehyde according to the invention and, as a comparative example, a mixture of methyl salicylate with benzyl benzoate with the refractive index adjusted to that of 2-hydroxy-5-methoxybenzaldehyde. The result of the clearing step was likewise photographed. The results are shown in FIG. 1, right-hand side. It was found that clearing with methyl salicylate/benzyl benzoate already results in good transparency in the tissue sample. However, the transparency achieved with the 2-hydroxy-5-methoxybenzaldehyde of the invention was even better still.

### Example 2

Several tissue samples (pig lung) having a volume of about 0.25 ml (0.5×0.5×1 cm 3) were prepared as described in example 1 using 2-hydroxy-5-methoxybenzaldehyde, ethyl cinnamate, and dibenzyl ether as embedding media.

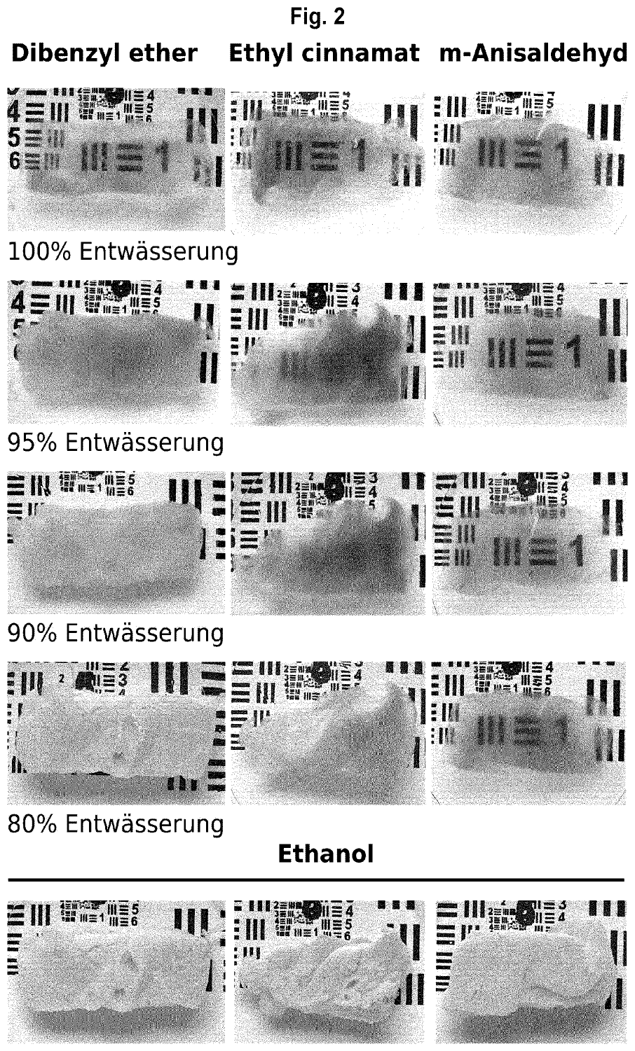

The tissue samples were then stored in absolute ethanol overnight and the embedding medium thus washed out of the tissue samples again. The tissue samples were transferred to ethanol having a defined water content so as to achieve a defined residual water content in the tissue, incubated for 2 hours, and then placed back in the respective embedding medium to be photographed. The tissue samples were placed in ethanol having an ethanol content of 95% by volume, 90% by volume, and 80% by volume. The results were photographed and are shown in FIG. 2. The first row shows the 100% dehydrated tissue samples in the embedding media, the second row shows the tissue samples incubated in ethanol having a water content of 5% by volume in the embedding medium, the third row shows the tissue samples incubated in ethanol having a water content of 10% by volume in the embedding medium, and the fourth row shows the tissue samples incubated in ethanol having a water content of 20% in the embedding medium. The bottom row shows the same tissue samples in absolute ethanol, i.e. before they had been placed in the respective embedding media. All samples are of equal opacity when completely dehydrated in ethanol.

It was found that the embedding media dibenzyl ether and ethyl cinnamate known from the prior art achieve the desired transparency only when there is no residual water or only a very small amount of residual water. In the case of DBE, a residual water content of 5% by volume is sufficient for the sample to no longer be transparent and therefore unable to be examined under the microscope. At a residual water content of 95% ECi likewise already shows clear cloudiness, and the tissue sample is no longer completely transparent. On the other hand, the tissue samples cleared with 2-hydroxy-5-methoxybenzaldehyde remain completely transparent even at a residual water content of 10% by volume (90% by volume ethanol) and show slight clouding only at 20% residual water (80% ethanol content).

### Example 3

Tissue samples having a volume of about 0.25 ml (0.5×0.5×1 cm 3) were dehydrated in absolute ethanol. They were then transferred to an embedding medium in a sample vessel. The embedding media used were 2-hydroxy-5-methoxybenzaldehyde according to the invention and, as a comparative example, dibenzyl ether and mixtures of methyl salicylate with benzyl benzoate. It was observed that in the case of 2-hydroxy-5-methoxybenzaldehyde the tissue sample had already sunk distinctly below the surface after 15 min and, depending on the tissue type, was lying on the bottom of the vessel after 30-45 min. In dibenzyl ether the tissue sample took several hours to sink to the bottom, comparable to the time required for mixtures of methyl salicylate/benzyl benzoate and DBE.

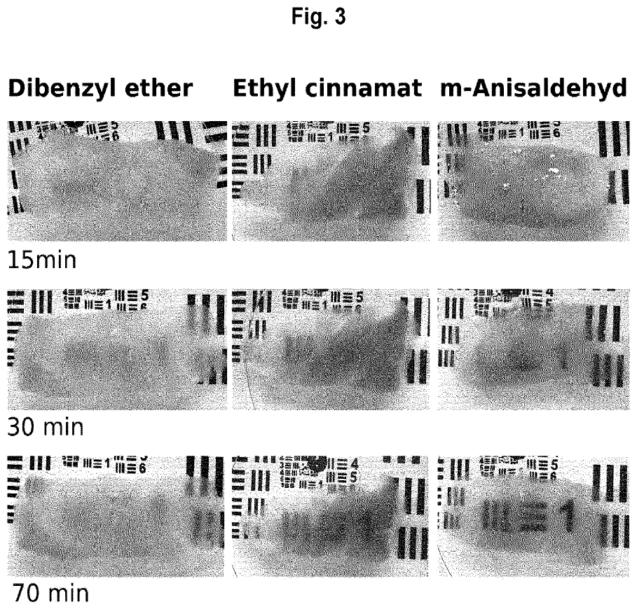

In a further experiment, the rate at which the dehydrating solvent ethanol in a tissue sample was replaced by the embedding media was determined. The embedding media used were 2-hydroxy-5-methoxybenzaldehyde according to the invention and dibenzyl ether and ethyl cinnamate as comparative examples. The tissue samples were placed in the embedding medium for 15 min, 30 min, and 70 min and then photographed. The results are shown in FIG. 3. In the top row, the tissue samples have been photographed after clearing for 15 min. In the middle row, the tissue samples have been photographed after clearing for 30 min. In the bottom row, the tissue samples have been photographed after clearing for 70 min. It can be seen that the tissue sample treated with 2-hydroxy-5-methoxybenzaldehyde is already transparent after 70 min, whereas the sample treated with ECi shows only slight transparency and the tissue sample treated with DBE is not yet transparent at all. The clearing method according to the invention thus achieves much more rapid clearing of the tissue sample.

The invention is not restricted to one of the embodiments described above, but may be modified in a variety of ways.

All the features and advantages apparent from the claims, the description, and the drawing, including method steps, may be essential to the invention either on their own or in the diversity of combinations.

1. A method for preparing transparent tissue samples of a biological tissue for examination by light microscopy, the method comprising the steps of:
a) dehydrating the tissue sample with a dehydrating solvent and
b) clearing the dehydrated tissue sample by placing it in an embedding medium comprising a benzaldehyde anisole ether selected from 3-methoxybenzaldehyde, 4-methoxybenzaldehyde, 2-hydroxy-5-methoxybenzaldehyde, and 4-ethoxybenzaldehyde.

2. The method as claimed in claim 1, wherein the embedding medium contains 10% to 100% by volume of the benzaldehyde anisole ether and 0% to 90% by volume of an optically suitable, inert organic solvent having a refractive index of about 1.3, preferably 1.5.

3. The method as claimed in claim 1, wherein the embedding medium contains 10% to 100% by volume of the benzaldehyde anisole ether and 0% to 90% by volume of an optically suitable, inert organic solvent having a refractive index of about 2.0, preferably 1.65.

4. The method as claimed in claim 1, wherein the embedding medium consists of a benzaldehyde anisole ether selected from 3-methoxybenzaldehyde, 4-methoxybenzaldehyde, 2-hydroxy-5-methoxybenzaldehyde, and 4-ethoxybenzaldehyde.

5. The method as claimed in claim 1, wherein the dehydration step a) involves the use of dehydrating compositions composed of aqueous alcohol, ketone or ether and that the dehydrating compositions is an aqueous mixture of an alcohol, ketone or ether in which the solvent concentration of the dehydrating compositions ranges from 50% to 98% by volume, preferably 70% to 98%, more preferably 75% to 98% by volume, an aqueous mixture of aqueous ethanol having increasing concentrations of ethanol, the ethanol concentrations of the dehydrating compositions ranging from 50% to 98% by volume.

6. The method as claimed in claim 1, wherein the dehydration step step a) is carried out in a gradient mixer and that the tissue sample is at the start of the dehydration step introduced directly into a dehydrating solvent having a solvent concentration of 50% by volume and the gradient is then increased in small increments.

7. The method as claimed in claim 1, wherein the tissue sample, before it is dehydrated in step a) and optically cleared in step b), at least one of:
is fixed with formaldehyde
is washed
is washed with water
is incubated in an aqueous alkaline solution
is delipidated with a detergent solution
is delipidated with an organic solvent
is bleached with oxidizing reagents or
is decolorized with amino alcohols.

8. The method as claimed in claim 1, wherein the tissue sample, before it is dehydrated in step a) and optically cleared in step b), is fixed and that the fixing agent is selected from
crosslinking fixatives such as formaldehyde, glutaraldehyde, acrolein, carbodiimides, diethyl pyrocarbonate, bisimidoesters or glyoxal or mixtures thereof, and/or
coagulant fixatives such as alcohols and other organic solvents, acids, potassium dichromate, lead nitrate, copper sulfate, and mercuric chloride and mixtures thereof.

9. The method as claimed in claim 1, wherein the optically cleared tissue sample is in a further step examined under a microscope in order to obtain an image of the internal structure of the sample, the microscope being a light microscope.

10. A kit for preparing biological tissue samples for light microscopy, the kit comprising:
a dehydrating solvent for dehydrating the tissue sample and
an embedding medium for clearing the dehydrated tissue sample by placing the sample in the embedding medium,
wherein the dehydrating solvent is an alcohol, ketone or ether and
the embedding medium is a benzaldehyde anisole ether selected from 3-methoxybenzaldehyde, 4-methoxybenzaldehyde, 2-hydroxy-5-methoxybenzaldehyde, and 4-ethoxybenzaldehyde.

11. The kit as claimed in claim 10, wherein the benzaldehyde anisole ether is selected from 3-methoxybenzaldehyde, 4-methoxybenzaldehyde, 2-hydroxy-5-methoxybenzaldehyde, and 4-ethoxybenzaldehyde.

12. The kit as claimed in claim 10, wherein the dehydrating solvent is selected from ethanol, methanol, tetrahydrofuran, isopropanol, tert-butanol, 2,2′-thiodiethanol, trichloroethanol, and acetone.

13. The method for use of a benzaldehyde anisole ether selected from 3-methoxybenzaldehyde, 4-methoxybenzaldehyde, 2-hydroxy-5-methoxybenzaldehyde, and 4-ethoxybenzaldehyde
as an embedding medium for preparing a biological tissue sample for examination by light microscopy.
